(19) United States
(12) Patent Application Publication
(10) Pub. No.: US 2025/0075246 A1
(43) Pub. Date: Mar. 6, 2025

(54) A BIOASSAY MODULE, A KIT FOR THE DETECTION OF BACILLUS CEREUS AND METHODS THEREOF

(71) Applicant: THE STATE OF ISRAEL, MINISTRY OF AGRICULTURE & RURAL DEVELOPMENT, AGRI-CULTURAL RESEARCH ORGANIZATIO, Rishon Lezion (IL)

(72) Inventors: Evgeni ELTZOV, Ashdod (IL); Manpreet KAUR, Rishon Lezion (IL)

(21) Appl. No.: 18/726,562

(22) PCT Filed: Jan. 3, 2023
(86) PCT No.: PCT/IL2023/050007

§ 371 Date: Jul. 3, 2024

Related U.S. Application Data
(60) Provisional application No. 63/296,922, filed on Jan. 6, 2022.

(51) Int. Cl. C12Q 1/04; G01N 21/78

(52) U.S. Cl. CPC C12Q 1/04; G01N 21/78

(57) ABSTRACT

The present invention teaches a bioassay module for detection of pathogenic microorganisms comprising: food specimen deposit element; indicator layer comprising dye; enzymatically digestible substrate layer, forming a barrier between the food specimen deposit element and indicator layer; isolation layer, separating the enzymatically digestible substrate layer from layers underlying the enzymatically digestible substrate layer; separation layer; absorption layer to absorb and display the indicator; wherein the layers are disposed from bottom to top the absorption layer, the separation layer, the indicator layer, the isolation layer, the enzymatically digestible substrate layer, the food specimen deposit element, and enzymatically digestible substrate layer is liquefiable and perforable by enzymes secreted by the pathogenic microorganism in contaminated food disposed in the food specimen deposit element, the digested substrate layer is flowable to the indicator layer via the isolating layer, the liquefied dye flowable to the absorption layer and visible at least from the top layer.

### FIELD OF INVENTION

The present disclosure relates to agro-food industries and food safety, more particularly to the detection and analysis of pathogenic microorganism by the point of care testing (POCT).

### BACKGROUND OF INVENTION

Bacillus cereus, a gram-positive spore forming bacteria, is deemed as the major cause of food poisoning (www.fsai.ie/WorkArea/DownloadAsset.aspx?id=10919). It is a soil bacterium that is majorly isolated from the foods of plant origin, however, owing to its ubiquitous nature and resistance of its endospores to extreme physical stresses, it frequently inhabits other foods types, viz., meat, eggs, dairy products and processed foods (Tewari A., Abdullah S., J. Food Sci. Technol.; 52:2500-2511, 2015). Food poisoning associated with B. cereus is categorized in two forms. The emetic form is caused by the ingestion of food contaminated with cereulide toxin produced by bacteria, resulting in vomiting and nausea. While the other form is caused by enterotoxins produced by B. cereus in the small intestine and is characterized by diarrhea and abdominal pain. Annual reports of the European Food Safety Authority (EFSA) have shown that approximately 3.9-5.5% of all annual food poisoning outbreaks from 2011-2015 were associated with B. cereus (Jessberger, N., Dietrich, R., Granum, P. E., & Märtlbauer, E. Toxins, 12(11), 1-37, 2020). Currently, molecular methods that are being employed for the detection of B. cereus are primarily based on polymerase chain reaction (PCR), viz., qPCR/Real-Time PCR, nested PCR, etc., as they are highly specific and allow rapid identification of the bacteria, however, these methods are labor-intensive, costly, require specialized equipment (thermal cycler) and skilled personnel to ensure accurate testing [(Hall, A. et. al, Food Control; 27:127-131, 2012) and (Fernández-No, I., et. al, Food Microbiol.; 28:605-610, 2011)]. Further, they have long turnaround time (24-48 h) and also have higher probability of giving false-negative results, hence, they cannot be used as a point-of-care (POC) detection system for food safety analysis in agro-food industries. Recently, biosensors are being considered as promising and economical methods for point-of-care diagnosis owing to their easy usability, higher efficacy, lower cost and high reliability and reproducibility. Various types of DNA-based and antibody-based biosensors have been employed for the detection of B. cereus, however, they suffer from a few limitations. High cost, low specificity and sensitivity of commercial antibodies to temperature affect their stability and binding capacity [(Ramarao, N., Tran, S. L., Marin, M., & Vidic, J., Sensors (Switzerland), 20(9), 2020) and (Vidic, J., Vizzini, P., et. al An Update Look. Micromachines, 10, 500, and Sensors, 19, 1100, 2019)]. Similarly, the efficiency of DNA-based biosensors can get affected by variations in pH or temperature and therefore specific storage conditions (buffers/media) are required to avoid the degradation of DNA and maintain its stability. Further, the electrochemical and optical DNA biosensors also require specialized devices for conducting the assays and for collecting/analyzing the data (Peña-Bahamonde, J., Nguyen, H. N., Fanourakis, S. K., & Rodrigues, D. F. Journal of Nanobiotechnology, 16(1), 1-17, 2018).

Therefore, in the context of the limitations of the existing detection methods, there is still an unmet long felt need to develop an easy to use, low-cost POCT device that excludes the need of employment of expensive devices/skilled staff and which allows the detection of B. cereus in a single-step.

In view of the prior art and given the various challenges described above, there is still an unmet long-felt need to

### SUMMARY OF THE INVENTION

It is thus one object of the present invention to disclose a bioassay module (10) for the detection of pathogenic microorganisms comprising:
a. food specimen deposit element (5),
b. an indicator layer (3) comprising dye,
c. an enzymatically digestible substrate layer (4), forming a barrier between said food specimen deposit element (5) and said indicator layer (3),
d. an isolation layer (6), for separating said enzymatically digestible substrate layer (4) from layers underlying said enzymatically digestible substrate layer,
e. a separation layer (2); and
f. an absorption layer to absorb and display said indicator (1),
   wherein the layers are disposed from bottom to top, the absorption layer (1), the separation layer (2), the indicator layer (3), the isolation layer (6), the enzymatically digestible substrate layer (4), the food specimen deposit element (5), and enzymatically digestible substrate layer is liquefiable and perforable by enzymes secreted by the pathogenic microorganism in contaminated food disposed in the food specimen deposit element (5), the digested substrate layer (4) is flowable to the indicator layer (3) via the isolating layer, the liquefied dye flowable to the absorption layer (1) and visible at least from the top layer.

It is another object of the present invention to disclose the aforementioned bioassay module (10), wherein the pathogenic microorganisms are selected from a group comprising Bacillus cereus, Enterococcus faecalis, Pseudomonas aeruginosa, Serratia marcescens, Bacillus amyloliquefaciens, Bacillus subtilis, Bacillus megaterium, Bacillus pichinotyi, Bacillus pumilus, Staphylococcus hominis, Alternaria alternate, Aspargilis ustus, Cladosporium cladosporioides, Penicillium chrysogemim, Aspargilis nidulans, Mucor racemosus, Trichoderma longibrachiatum, Aspargilis versicolor, Cryptococcus albidus and any combination thereof.

It is another object of the present invention to disclose the bioassay module (10) as defined above, wherein the aforementioned enzymatically digestible substrate layer (4) is a bacteria specific substrate.

It is thus another object of the present invention to disclose the bioassay module (10) as defined above, wherein the aforementioned enzymatically digestible substrate layer (4) is selected from a group comprising gelatin, agar and PVA (Poly(vinyl alcohol)).

It is thus another object of the present invention to disclose the bioassay module (10) as defined above, wherein the aforementioned enzymatically digestible substrate layer (4) is a gelatin layer.

It is thus another object of the present invention to disclose the bioassay module (10) as defined above, wherein the aforementioned enzymatically digestible substrate layer (4) is a combination of support material selected from a group comprising alginate, sol-gel, gelatin and any combination thereof and an bacteria specific enzymatically digestible substrate.

It is thus another object of the present invention to disclose the bioassay module (10) as defined above, wherein the aforementioned food specimen deposit element (5) allows a food specimen to be placed on top of the aforementioned enzymatically digestible substrate layer and prevents spillage.

It is thus another object of the present invention to disclose the bioassay module (10) as defined above, wherein the aforementioned indicator layer (3) is selected from a group consisting, color indicator, light indicator, pH indicator, consistency indicator, electrochemical indicator and any combination thereof.

It is thus another object of the present invention to disclose a kit for the detection of pathogenic microorganisms in food comprising:
a. a food specimen deposit element (5),
b. an indicator layer (3),
c. ingredients for the preparation of an enzymatically digestible substrate layer (4),
d. an isolation layer (6),
e. a separation layer (2); and
f. an absorption layer (1),
g. preparation, assembly and operation instructions,
   wherein consequent to the preparation of the enzymatically digestible substrate layer according to the instructions, the layers and the food specimen deposit element are assembled and layered from bottom to top, the absorption layer (1), the separation layer (2), the indicator layer (3), the isolation layer (6), the enzymatically digestible substrate layer (4) and the food specimen deposit element (5), once all layers are assembled a food specimen is deposited for the detection of said pathogenic microorganisms.

It is yet another object of the present invention to disclose the bioassay kit, as defined above, wherein the aforementioned pathogenic microorganisms are selected from a group comprising Bacillus cereus, Enterococcus faecalis, Pseudomonas aeruginosa, Serratia marcescens, Bacillus amyloliquefaciens, Bacillus subtilis, Bacillus megaterium, Bacillus pichinotyi, Bacillus pumilus, Staphylococcus hominis, Alternaria alternate, Aspargilis ustus, Cladosporium cladosporioides, Penicillium chrysogemum, Aspargilis nidulans, Mucor racemosus, Trichoderma longibrachiatum, Aspargilis versicolor, Cryptococcus albidus and any combination thereof.

It is yet another object of the present invention to disclose the bioassay kit as defined above, wherein the aforementioned enzymatically digestible substrate layer (4) is a bacteria specific substrate.

It is yet another object of the present invention to disclose the bioassay kit as defined above, wherein the aforementioned enzymatically digestible substrate layer (4) is selected from a group comprising gelatin, agar and PVA (Poly(vinyl alcohol)).

It is yet another object of the present invention to disclose the bioassay kit as defined above, wherein the aforementioned enzymatically digestible substrate layer (4) is a gelatin layer.

It is yet another object of the present invention to disclose the bioassay kit as defined above, wherein the aforementioned enzymatically digestible substrate layer (4) is a combination of support material selected from a group comprising alginate, sol-gel, gelatin and any combination thereof and an bacteria specific enzymatically digestible substrate.

It is yet another object of the present invention to disclose the bioassay kit as defined above, wherein the aforementioned food specimen deposit element (5) allows a food specimen to be placed on top of the aforementioned enzymatically digestible substrate layer (4) and prevents spillage.

It is yet another object of the present invention to disclose the bioassay kit as defined, wherein the aforementioned indicator layer (3) is selected from a group consisting, color indicator, light indicator, pH indicator, consistency indicator, electrochemical indicator and any combination thereof.

It is yet another object of the present invention to disclose a method for the detection of food pathogenic microorganisms comprising:
a. obtaining a bioassay module (10) according to claim 1,
b. depositing food specimen in said food specimen deposit element (5),
c. monitoring said indicator dye on said absorption layer (1).

It is yet another object of the present invention to disclose the method as defined above, wherein the aforementioned pathogenic microorganisms are selected from a group comprising Bacillus cereus, Enterococcus faecalis, Pseudomonas aeruginosa, Serratia marcescens, Bacillus amyloliquefaciens, Bacillus subtilis, Bacillus megaterium, Bacillus pichinotyi, Bacillus pumilus, Staphylococcus hominis, Alternaria alternate, Aspargilis ustus, Cladosporium cladosporioides, Penicillium chrysogemim, Aspargilis nidulans, Mucor racemosus, Trichoderma longibrachiatum, Aspargilis versicolor, Cryptococcus albidus and any combination thereof.

It is yet another object of the present invention to disclose the method as defined above, wherein the aforementioned enzymatically digestible substrate layer (4) is a bacteria specific substrate.

It is yet another object of the present invention to disclose the method as defined above, wherein the aforementioned enzymatically digestible substrate layer (4) is selected from a group comprising gelatin, agar and PVA (Poly(vinyl alcohol)).

It is yet another object of the present invention to disclose the method as defined, wherein the aforementioned enzymatically digestible substrate layer (4) is a gelatin layer.

It is yet another object of the present invention to disclose the method as defined, wherein the aforementioned enzymatically digestible substrate layer (4) is a combination of support material selected from a group comprising alginate, sol-gel, gelatin and any combination thereof and an bacteria specific enzymatically digestible substrate.

It is yet another object of the present invention to disclose the method as defined above, wherein the aforementioned food specimen deposit element (5) allows a food specimen to be placed on top of the aforementioned enzymatically digestible substrate layer and prevents spillage.

It is yet another object of the present invention to disclose the method as defined, wherein the aforementioned indicator layer (3) is selected from a group consisting, color indicator, light indicator, pH indicator, consistency indicator, electrochemical indicator and any combination thereof.

It is yet another object of the present invention to disclose a method for the detection of food poisoning related microorganisms comprising:
a. obtaining a kit according to claim 8,
b. preparing and assembling said kit according to said instructions
c. depositing a food specimen in said food specimen deposit element (5),
d. monitoring said indicator on said absorption layer (1).

It is yet another object of the present invention to disclose the method as defined, wherein the aforementioned pathogenic microorganisms are selected from a group comprising Bacillus cereus, Enterococcus faecalis, Pseudomonas aeruginosa, Serratia marcescens, Bacillus amyloliquefaciens, Bacillus subtilis, Bacillus megaterium, Bacillus pichinotyi, Bacillus pumilus, Staphylococcus hominis, Alternaria alternate, Aspargilis ustus, Cladosporium cladosporioides, Penicillium chrysogemum, Aspargilis nidulans, Mucor racemosus, Trichoderma longibrachiatum, Aspargilis versicolor, Cryptococcus albidus and any combination thereof.

The method as defined above, wherein the aforementioned enzymatically digestible substrate layer (4) is a bacteria specific substrate.

It is yet another object of the present invention to disclose the method as defined above, wherein the aforementioned enzymatically digestible substrate layer (4) is selected from a group comprising gelatin, agar and PVA (Poly(vinyl alcohol)).

It is yet another object of the present invention to disclose the method as defined above, wherein said enzymatically digestible substrate layer (4) is a gelatin layer.

It is yet another object of the present invention to disclose the method as defined above, wherein the aforementioned enzymatically digestible substrate layer (4) is a combination of support material selected from a group comprising alginate, sol-gel, gelatin and any combination thereof and an bacteria specific enzymatically digestible substrate.

It is yet another object of the present invention to disclose the method as defined above, wherein the aforementioned food specimen deposit element (5) allows a food specimen to be placed on top of the aforementioned enzymatically digestible substrate layer and prevents spillage.

It is yet another object of the present invention to disclose the method as defined above, wherein the aforementioned indicator layer (3) is selected from a group consisting, color indicator, light indicator, pH indicator, consistency indicator, electrochemical indicator and any combination thereof.

These exemplary embodiments are mentioned not to limit or define the invention, but to provide examples of embodiments of the invention to aid understanding thereof. Exemplary embodiments are discussed in the Detailed Description, and further description of the invention is provided there. Advantages offered by the various embodiments of the present invention may be further understood by examining this specification.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention.

These and other features, aspects, and advantages of the present invention are better understood when the following Detailed Description is read with reference to the accompanying drawings, wherein:

FIG. 1a-1c depicting a schematic representation of the bioassay module for detection of B. cereus and the different stages.

FIG. 2a-2e depicting a schematic representation of different stages of the liquification of the gelatin layer of the bioassay module for detection of B. cereus.

FIG. 3a-3b depicting the layers of the bioassay module separately (FIG. 3a) and the assembled module of the bioassay (FIG. 3b).

FIG. 4a-4c graphic presentation of the effect of (3a) 6% (3b) 8% and (3c) 10% concentration of gelatin on the hydrolysis of gelatin layer with respect to liquefaction time.

FIG. 5a depicting the specificity of the bioassay module for different bacterial strains.

FIG. 5b depicting bioassay modules injected with different bacterial strains (Image taken after 10 h).

FIG. 6 depicts Liquefaction time v. B. Cereus CFU, demonstrating assay sensitivity.

FIG. 7 depicts results of the assay comparing different food types.

### DETAILED DESCRIPTION OF PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a bioassay module, a kit and methods for the identification of B. cereus in food samples based on an enzymatic substrate digestion, performed by B. cereus secreted enzymes.

As used hereinafter, the term “point-of-care testing (POCT)” refers to a test or an assay performed in a location of interest, e.g., a food factory, a food serving institute etc. and without the need to send a sample to a laboratory. Instead, POC testing makes it far easier for results to be obtained quickly and reliably. With these results available, professional staff can more easily make informed decisions about a patient's treatment and care.

As used hereinafter, the term “polymerase chain reaction (PCR)” refers to a molecular biology technique for DNA amplification at times, even from a very small amount of DNA. PCR is a common tool for DNA sequencing, for detecting the presence or absence of a gene to help identify pathogens during infection, and when generating forensic DNA profiles from tiny samples of DNA.

Each PCR procedure requires:
1. The DNA template to be amplified.
2. Primers, short stretches of DNA that initiate the PCR reaction, designed to bind to either side of the section of DNA to be amplified.
3. DNA nucleotide bases (dNTPs). DNA bases (A, C, G and T) are needed to construct the new strands of DNA.
4. Taq polymerase enzyme to add in the new DNA bases to the newly polymerized DNA strand.
5. Buffer to ensure the right conditions for the reaction.

PCR involves a process of heating and cooling called thermal cycling which is carried out by machine. The thermal cycle provides the right conditions for the three main stages of the PCR, Denaturing (the double-strand DNA is heated to separate it into two single strands), Annealing (the temperature is lowered to enabling the primers to attach to the template DNA). and Extending (temperature is raised to allow the Taq polymerase to polymerize the new DNA strand over a template) the thermal cycle may be repeated many times.

As used hereinafter, the term “Quantitative PCR (qPCR)” refers to a method for the detection, quantification, and typing of different microbial agents in the areas of molecular biology, clinical and veterinary diagnostics and food safety, also known as Real-Time PCR. Generally, in qPCR one is monitoring DNA amplification in real-time through the monitoring of fluorescence. This same principle of amplification of PCR is employed in qPCR/Real-Time PCR. But instead of looking at bands on a gel at the end of the reaction, the process is monitored in “real-time”. The reaction is placed into a qPCR/real-time PCR machine that watches the reaction occur with a camera or detector. In qPCR, fluorescent labeling enables the collection of data as PCR progresses. This technique has many benefits due to the range of methods and chemistries available.

Although many different techniques are used to monitor the progress of a PCR reaction, all have one thing in common. They all link the amplification of DNA to the generation of fluorescence which can simply be detected with a camera during each PCR cycle. Hence, as the number of gene copies increases during the reaction, so does the fluorescence, indicating the progress of the reaction

In qPCR/real time PCR, fluorescence is measured after each cycle and the intensity of the fluorescent signal reflects the momentary amount of DNA amplicons in the sample at that specific time. In initial cycles the fluorescence is too low to be distinguishable from the background. However, the point at which the fluorescence intensity increases above the detectable level corresponds proportionally to the initial number of template DNA molecules in the sample. This point is called the quantification cycle (C_{q}) and allows determination of the absolute quantity of target DNA in the sample.

There are two strategies for the real time visualization of amplified DNA fragments—non-specific fluorescent DNA dyes and fluorescently labeled oligonucleotide probes. These two approaches are used in pathogen detection.

The PCR is an exponential process where the number of DNA molecules theoretically doubles after each cycle. More generally, the amplification reaction follows this equation:

$${N}_{n} = {N}_{0} \times {( 1 + E )}^{n}$$

where Nₙ is the number of PCR amplicons after n cycles, N₀ is the initial number of template copies in the sample, E is the PCR efficiency that can assume values in the range from 0 to 1 (0-100%) and n is number of cycles. The PCR efficiency is therefore a significant factor for the quantification of the target DNA in unknown samples. The results are compared with a calibration curve, determined by spacing of known number of DNA copies serial dilutions, thus, enabling quantification.

As used herein after, the term “nested PCR” refers to a modification of PCR designed to improve sensitivity, specificity and yield. In this method, two pairs of PCR primers are designed: one set (outer primers/upstream) flanks a region of DNA containing the amplicon of interest, while a second set (nested primers) corresponds to the precise region of DNA to be amplified. The outer/upstream primers are used in a first PCR reaction to amplify the target with extended flanking regions. The product of this first PCR reaction then serves as a template in a second PCR reaction with only the nested primers. In the event that nonspecific products are amplified during the first PCR reaction, it is very unlikely that the same nonspecific region would be recognized and sequentially amplified by the second primer set, so specificity is still promoted by the second set of primers if they recognize the intended target. Another benefit of two PCR reactions is that this approach helps to obtain a sufficient yield of the desired target from a limited amount of input DNA As used hereinafter, the term “limit of detection (LOD)” refers to the lowest concentration of a component that can be measured (detected) with statistical significance by means of a given analytical procedure.

Bacillus cereus is recognized as a major foodborne pathogen that causes food poisoning and other types of infections. The present invention discloses a gelatin-based assay for the rapid detection of B. cereus in food to mitigate the risk of contamination. The disclosed multi-layer bioassay module includes the incorporation of a gelatin film of optimal concentration (10%) that gets liquefied owing to the gelatinase activity of B. cereus. The liquefied sample passes through the colored layer causing the dye to migrate towards the bottom-placed absorption layer, thereby generating a positive colorimetric signal. The assay was able to detect B. cereus in the range of 10²-10³ CFU/mL in different food samples, thereby exhibiting the higher or comparable sensitivity to the available methods of detection, which relies on the usage of expensive procedures. Hence, the developed assay represents a low-cost, simple, and portable point-of-care method to sensitively detect B. cereus in food specimens.

The disclosed system is based on the use of synthetic substrate-enzyme reaction for the identification of B. cereus, which would allow for the detection of specific enzymatic activity of B. cereus. B. cereus possesses gelatinase activity and is highly active in degrading the gelatin into smaller polypeptides, peptides, and amino acids that can be utilized by the organism (Abfalter, C., Schmidt, T., & Wessler, S. British Microbiology Research Journal, 7(2), 62-70, 2015).

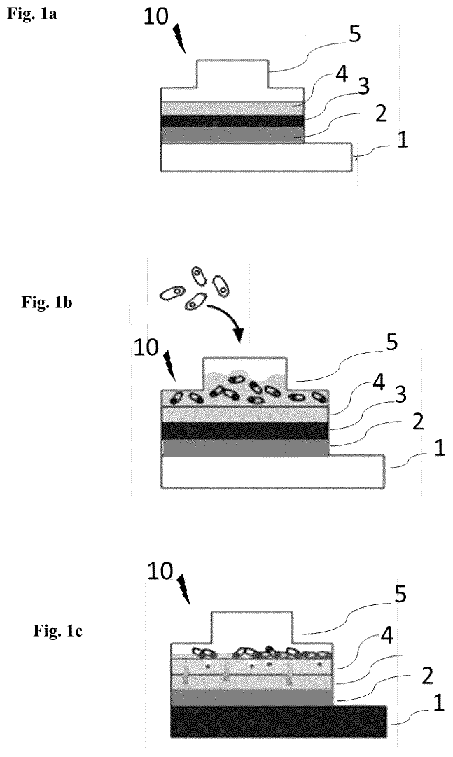

Reference is now made to a preferred embodiment of the present invention, FIG. 1a depicting the bioassay module for the detection of B. cereus, with a gelatin layer, used as a substrate in the detection kit printed by 3D printing (10). The bioassay module comprises an absorption layer (1), an isolating layer (2) colored layer (3), a gelatin layer (4), and a 3D-printed sample holder (5) for sample injection. When a sample infected with B. cereus is injected into this bioassay module (FIG. 1b), the sample migrates to the gelatin layer, where the bacteria start secreting the gelatinase enzyme, which liquefies the gelatin. As the liquefied sample passes through the colored layer (FIG. 1c), it causes migration of the dye towards the bottom-placed absorption layer, thereby giving a positive colorimetric signal.

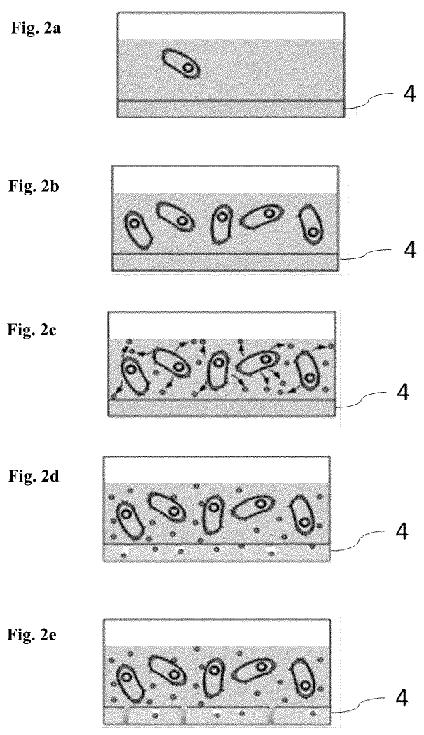

Reference is now made to FIGS. 2a-e, focusing only on the gelatin layer (4) and the Bacteria sample. A sample containing a low concentration of B. cereus is put on top of the gelatin layer (FIG. 2a), the bacteria reproduce and the B. cereus concentration is rising (FIG. 2b), as the concentration of secreted gelatinase enzyme (FIG. 2c), the gelatinase begins to exert its activity (FIG. 2d), resulting in the liquefying of the gelatin layer by the gelatinase (FIG. 2e). Subsequently, the liquified mass passes through the colored layer, causing the dye to migrate to the absorption layer giving a positive colorimetric signal (not shown).

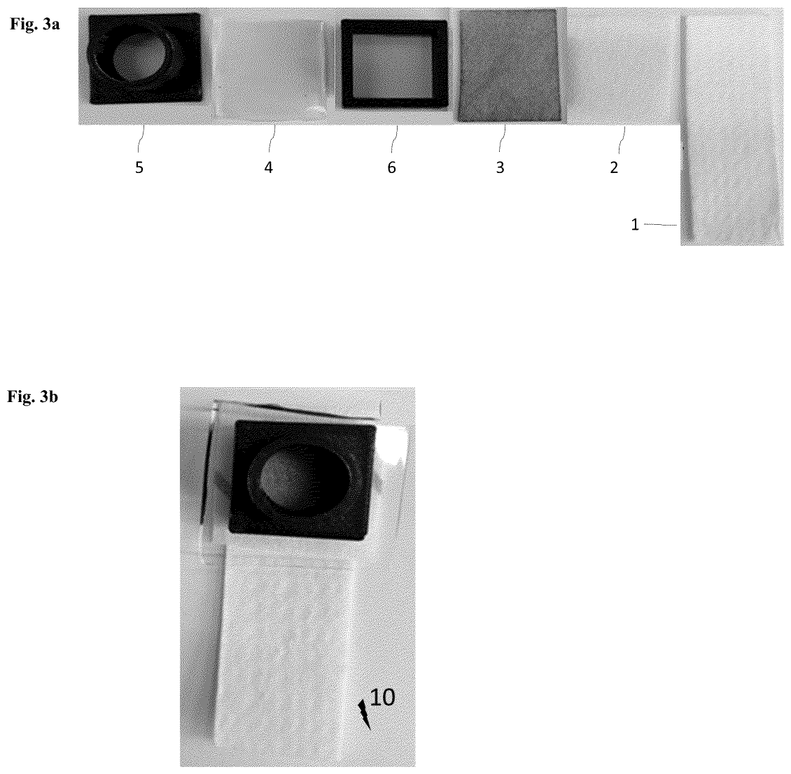

Reference is now made to FIG. 3a-3b, depicting the layers of the bioassay module separately (FIG. 3a) and the assembled bioassay module (FIG. 3b). the bioassay module was assembled by stacking all layers, one on top of the other, in the order (from bottom to top): absorption layer (1), separation layer (2), colored layer (3), isolating layer (6), gelatin layer (4), and sample plastic holder (5) (FIG. 3a). The dimensions of the absorption layer, separation layer, and colored layer were kept fixed for all modules, and the membrane layers were then stuck together with an adhesive tape (FIG. 3b). The purpose of the isolating layer is to separate the gelatin layer from the bottom layers (colored layer, separation layer and absorption layer) in order to avoid the absorption of gelatin moisture by those layers and avoid a false negative signal. The gelatin layer (4) was cut from the solidified gelatin plates using a scalpel and was then placed on the isolating layer (6). On top of the gelatin layer, a sample plastic holder (5) is placed, which assists in holding an optimal volume of sample on the gelatin layer, thereby preventing its spillage.

### EXAMPLES

### Example 1—Optimization of the Concentration and Thickness of the Gelatin Layer

Optimizing the concentration and thickness of the gelatin layer is crucial since it is directly related to the hydrolyzing time. During the formation of gelatin film, the single strands gelatin chains cross-linked to form a three-dimensional (3D) triple-helix network which is stabilized by hydrogen bonding and van der Waals interactions.

The degradation of a gelatin film may be attributed to two separate processes that may occur concurrently, dissolving and digestion; each has a different time rate. Dissolving is a ubiquitous a-biotic process and takes place through all gelatin surfaces that are in contact with a solution. However, digestions are specific to the presence of bacteria in contact with the gelatin, where they release the digesting enzymes. The digestion process is highly advantageous for the detection of bacterial presence and activity since a small puncture is enough to diffuse the sample and the liquified through the gelatin layer.

While, for liquefaction of high gelatin concentration, enhanced enzymatic secretion by bacteria is imperative to break the 3D matrix of gelatin strands, which may increase the hydrolyzing time, thereby leading to a longer turnaround time of detection. Further, if the sample settles on the surface of the gelatin layer for a prolonged duration, it can increase the risk of cross-contamination of the sample with other microbes. Hence, to determine the effect of gelatin concentration on the liquefaction time and on the enzymatic potential of bacteria, gelatin layers of three different concentrations (6, 8, and 10% (w/v)) were prepared. Similarly, as the thickness of the gelatin layer is also correlated to the hydrolyzing time hence, this key parameter was also evaluated using three types of gelatin layers (G-10, G-15, G-20) prepared with different volumes (10, 15, 20 ml) of gelatin solution for all the tested gelatin concentrations.

Gelatin films were prepared using three different concentrations of gelatin (6%, 8%, and 10% (w/v)). The gelatin was first weighed and then added into deionized water according to the required concentration. Once added, the solution was mixed on a hot plate at 250 rpm and 100° C. for 10 min to obtain a homogenous solution. Then, the dissolved gelatin solution was placed in disposable plastic Petri dishes in three different volumes (10 ml, 15 ml, and 20 ml) to prepare films of varying thickness. The plates were kept at 25° C. until the gelatin completely solidified.

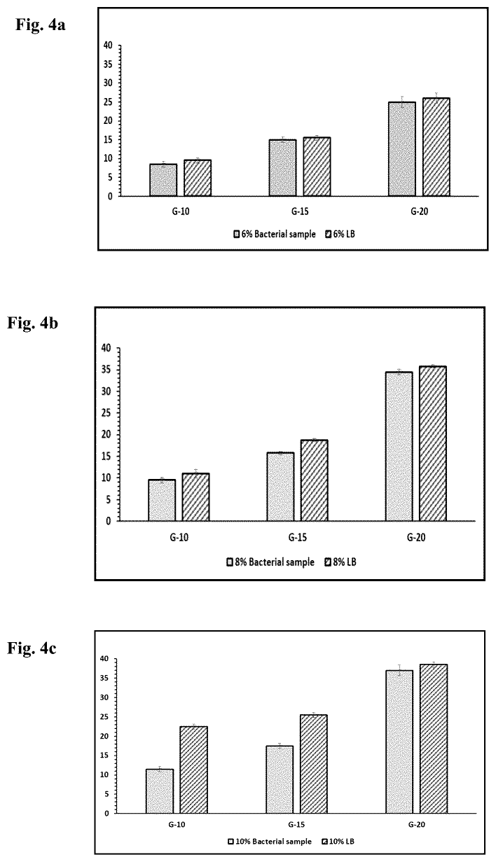

As the samples were applied on a G-10, G-15 and G-20, 6% (w/v) gelatin film, the difference in the liquefaction time of gelatin layers incubated with bacterial sample (8.5 h) and control sample (LB) (9.5 h) was noticed as insignificant, therefor G-10, G-15 and G-20, 6% (w/v) gelatin film were not further used. (FIG. 4a). Along similar lines, a minor difference in the liquefaction time was observed with 8% (w/v) gelatin films, G-10, G-15 and G-20, incubated with both bacterial (9.5 h, 15.5 h) and control (LB) samples (10.5 h, 18.5 h) (FIG. 4b). These results signify that the higher water content of the gelatin film increases the distance between gelatin chains, thereby reducing the number of polymer chains per unit volume which eventually results in poor mechanical strength. Hence, it can be concluded that the liquefaction of 6% and 8% gelatin films was not a result of bacterial enzymes but due to its increased sensitivity to water. However, with 10% (w/v) gelatin, a significant difference in the liquefaction time among the bacterial and control sample was observed, as the 10% (w/v) gelatin, G-10 layer was hydrolyzed completely with B. cereus-infected sample in 11.5 h, while flow-through with control samples (LB) was recorded at 22.5 h (FIG. 4c). This implies that the tensile strength and elasticity of the gelatin film increase with increasing concentration owing to a higher crosslinking degree.

To validate that the liquefaction of 10% (w/v) gelatin film was due to bacterial enzymes, it was noticed that bacterial enzymes concentrate at one area of gelatin film, thereby resulting a puncture from which the sample diffuses. While for 6% (w/v) and 8% (w/v) films, the water diffuses through the entire layer thickness fast enough to ensure that there is no vertical water content gradient in the polymer layer. Although with G-15 gelatin film of 10% (w/v) concentration, slightly higher hydrolyzing time was recorded for both bacterial samples (17 h) and control sample (26 h) compared to the G-10 film; however, the variance in the liquefaction time among bacterial-infected and control sample was quite substantial.

The late-stage hydrolysis of G-20 films of all gelatin concentrations occurs mainly due to the low hydration of the film, as it is well known that wetting by a droplet of a solvent on a soluble polymer substrate strongly depends on the degree of hydration of the polymer and that a dry layer can be poorly wetted by water even though the polymer is soluble in it.

The difference in the hydrolyzing time of G-20 films of all gelatin concentrations between bacterial-infected and control samples was also found to be insignificant (FIG. 4a-4c). This could be attributed to the colonization of the control sample by other microbes due to the extended incubation of the sample on the gelatin layer. Therefore, among the tested gelatin concentrations, 10% (w/v) concentration is preferred as the suitable concentration as owing to its firm and stable nature, it provides bacteria with optimal time to exhibit gelatinase activity and also decreases the likelihood of false-positive results. Further, among the 10% (w/v) gelatin films, the G-10 gelatin film is the most suitable as it resulted in a shorter detection time as compared to the G-15 film.

### Example 2—Specificity of the Bioassay Module

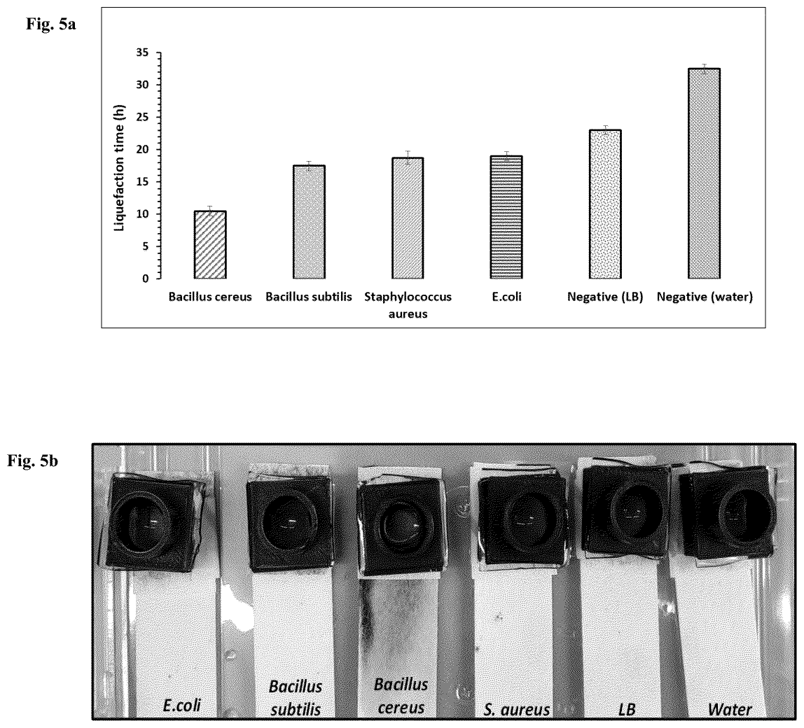

The specificity of the bioassay module for the detection of gelatinase-producing bacterial species were determined by employing the designed bioassay module with different bacterial strains: E. coli (DHα), S. typhimurium, B. cereus and B. subtilis (FIG. 5a-5b). For each experiment, bacterial strains were cultured in 20 ml freshly prepared LB and incubated overnight at 37° C. in a rotary thermo-shaker MaxQ 4450 (Thermo scientific (Marietta, OH, USA)) at 120 rpm. Then cultures were diluted with fresh LB to the early log phase of 10⁶ cell/mL (optical density at 600 nm (OD600 nm)=0.5) as determined by an ultrospec 2100 Pro spectrophotometer (Amersham Bioscience (Biochrom, Cambridge, England).

The bacterial solutions of the different strains with constant concentration (10⁶ cfu/mL) were disposed on top of the gelatin layer in the plastic holder, allowing the sample to diffuse from the top to bottom layers. Among all the bioassay modules, the flow-through was observed with B. cereus solution after 10.5 h, while with S. aureus, B. subtilis, and E. coli, the dissolution of gelatin layer was observed at 17.5 h, 18.5 h, and 19 h, respectively (see FIG. 5a-5b). While the bioassay module was incubated also with different control samples, i.e., LB and water showed the dissolution of gelatin layer at 23 h and 32.5 h, respectively. The differences in the dissolution time between the water control sample and LB control sample clearly signify that owing to the high nutrient content of LB, it is more prone to cross-contamination by other microbes, which facilitates early hydrolysis of gelatin layer incubated with LB as compared to water.

Although S. aureus, B. subtilis, and E. coli have been reported to possess gelatinase activity in various reports, the present application establishes that B. cereus exhibited maximum gelatinase activity as compared to S. aureus, B. subtilis, and E. coli bacterial strains, thereby leading to the detection of B. cereus in minimal possible time. Therefore, it can be concluded that though this bioassay module could be used for the identification of other gelatinase-positive pathogenic bacterial species, however, it may require some adjustment such as varied or longer detection time.

### Example 3—Sensitivity of the Bioassay Module

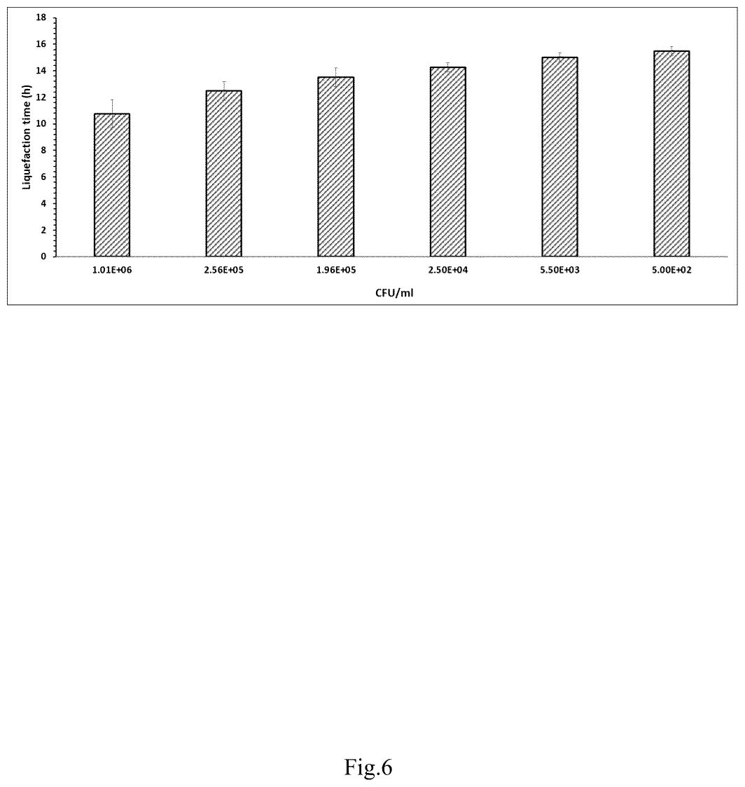

The sensitivity of the bioassay module was evaluated by layering B. cereus solutions of different concentrations (10², 10³, 10⁴, 10⁵, 10⁶ cfu/mL) on the gelatin layer; as the concentration of bacteria decreased, an increase in the liquefaction time of gelatin was observed (FIG. 6). The shorter liquefaction time of gelatin in higher bacterial concentrations (10⁴-10⁶ cfu/mL) is attributed to the larger amounts of secreted gelatinase by the larger number of bacteria. The larger amount of secreted gelatinase covers the surface of the gelatin layer in a much shorter time with a higher concentration of gelatinase at any given point, exerting its digestion effect faster and more efficiently, thereby leading to a rapid liquefaction of the gelatin layer. On the contrary, with lower bacterial concentration, it takes a relatively longer time for the bacteria to secrete enough gelatinase required for the liquefaction of gelatin film. However, the difference in liquefaction time of the gelatin layer incubated with 10² and 10³ cfu/mL bacterial concentrations was observed as insignificant.

### Example 4—Effect of Different Food Samples on the Bioassay Module Efficiency

To evaluate the potentiality of the bioassay module for the detection of B. cereus in food samples, the test was evaluated with three different food samples, viz., pasteurized milk, cooked rice. Three different food samples were tested: cooked rice, milk (pasteurized and 3% (w/v) fat content milk (Tnuva)). In order to keep the volume constant for each food sample, the cooked rice sample was first mashed in a grinder and diluted with double distilled water to a liquid consistency. The food samples were inoculated with varying concentrations of B. cereus cells (10², 10³ and 10⁶ cfu/mL) were placed into the bioassay module's plastic holder; uninfected food samples were used as control.

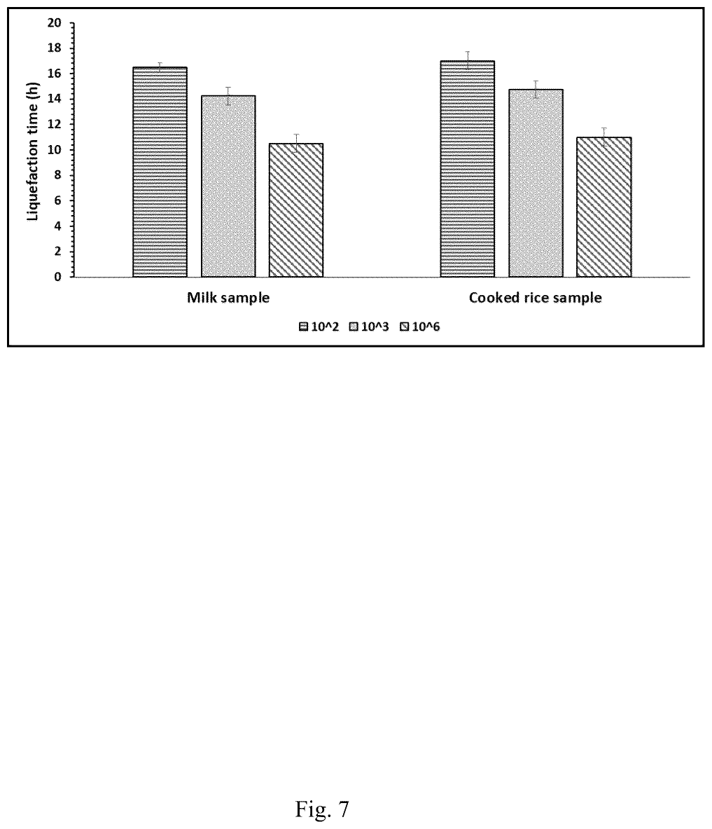

The assessment of inoculated food samples with varying concentrations of B. cereus (10², 10³, 10⁶ cfu/mL) clearly demonstrates the efficacious use of the gelatinase bioassay module for providing highly sensitive detection with the limit of detection (LOD) of 10² cfu/ml (FIG. 7). The results showed slight variation in the liquefaction time of the gelatin layer among the tested food samples, owing to the different textures, viscosities, and compositions of each sample. Among different food samples, flow-through of milk samples through the gelatin film to the absorbent layer was observed in comparatively less time as compared to the cooked rice sample. However, with all the un-inoculated food sample controls, diffusion of samples through the bioassay modules was not detected until 36 h. Hence, the proposed bioassay module may be efficiently employed as a fast and simple tool for monitoring a variety of foods infected with B. cereus.

The present invention discloses a bioassay module based on a substrate-enzymatic reaction for the identification of B. cereus in food samples. This invention employs the gelatinase activity of B. cereus, wherein a gelatin film of optimal concentration was placed in a simple bioassay module prepared with membrane layers and 3D printed modules. In the presence of pathogenic bacteria in the sample, the gelatin film is hydrolyzed by bacterial enzymes into a liquid that seeps through the colored membrane layer, thereby coloring the absorbent layer placed at the bottom of the bioassay module. This bioassay module and method were optimized and validated with different food samples that are prone to get contaminated by B. cereus. Despite being a very low-cost and easy-to-use bioassay module, kit and method, its sensitivity is highly comparable with other conventional methods currently being employed for the detection of B. cereus. Owing to the flexibility of this assay, it allows the identification of bacteria in a wide variety of food samples in food processing industries without the need for a diagnostic laboratory. Moreover, the bioassay module can be easily modified to detect different pathogens by employing bacteria specific substrates instead of gelatin.

Reference is now made to an embodiment of the present invention disclosing the bioassay module mentioned above, wherein the indicator may possess different indicating activity such as, but not limited to, color change, pH change, consistency change, light emission, electric characteristics change, etc.

Reference is now made to an embodiment of the present invention disclosing the bioassay module mentioned above, wherein the bioassay module is employed for the detection of different pathogenic microorganisms by substituting of the gelatin layer with any chemical that is a specific substrate for digesting extracellular enzymes, such as but not limited to, agar, PVA (Poly(vinyl alcohol)). Each substrate is specific to a certain bacterial species. The incorporation of different substrate layers within the designed bioassay module would combine the high sensitivity and selectivity of a specific enzymatic reaction associated with a particular bacterial species.

Reference is now made to an embodiment of the present invention disclosing the bioassay module mentioned above, wherein the enzymatically digestible layer is a combination of a support material, such as but not limited to alginate, sol-gel, gelatin and an enzymatically digestible substrate (i.g., modular system).

Reference is now made to an embodiment of the present invention disclosing a portable and low-cost kit for detection of pathogenic organisms in food, wherein the aforementioned kit can be operated by unskilled personnel in a location of interest for rapid determination of B. cereus or other pathogenic organisms in various types of food samples.

The invention is not intended to be limited to the embodiment illustrated and described above, but it can be modified and varied within the scope and spirit of the invention as defined by the following claims.

1.-32. (canceled)

33. A bioassay module (10) for the detection of pathogenic microorganisms comprising:
a. food specimen deposit element (5),
b. an indicator layer (3) comprising dye,
c. an enzymatically digestible substrate layer (4), forming a barrier between said food specimen deposit element (5) and said indicator layer (3),
d. an isolation layer (6), for separating said enzymatically digestible substrate layer (4) from layers underlying said enzymatically digestible substrate layer,
e. a separation layer (2); and
f. an absorption layer to absorb and display said indicator (1),
wherein said layers are disposed from bottom to top said absorption layer (1), said separation layer (2), said indicator layer (3), said isolation layer (6), said enzymatically digestible substrate layer (4), said food specimen deposit element (5), and enzymatically digestible substrate layer is liquefiable and perforable by enzymes secreted by said pathogenic microorganism in contaminated food disposed in said food specimen deposit element (5), said digested substrate layer (4) is flowable to said indicator layer (3) via said isolating layer, said liquefied dye flowable to said absorption layer (1) and visible at least from the top layer.

34. The bioassay module (10), according to claim 33, wherein said pathogenic microorganisms are selected from a group comprising Bacillus cereus, Enterococcus faecalis, Pseudomonas aeruginosa, Serratia marcescens, Bacillus amyloliquefaciens, Bacillus subtilis, Bacillus megaterium, Bacillus pichinotyi, Bacillus pumilus, Staphylococcus hominis, Alternaria alternate, Aspargilis ustus, Cladosporium cladosporioides, Penicillium chrysogenum, Aspargilis nidulans, Mucor racemosus, Trichoderma longibrachiatum, Aspargilis versicolor, Cryptococcus albidus and any combination thereof.

35. The bioassay module (10) according to claim 33, wherein said enzymatically digestible substrate layer (4) is a bacteria specific substrate.

36. The bioassay module (10) according to claim 33, wherein said enzymatically digestible substrate layer (4) is selected from a group comprising gelatin, agar and PVA (Poly(vinyl alcohol)).

37. The bioassay module (10) according to claim 33, wherein said enzymatically digestible substrate layer (4) is a gelatin layer.

38. The bioassay module (10) according to claim 33, wherein said enzymatically digestible substrate layer (4) is a combination of support material selected from a group comprising alginate, sol-gel, gelatin and any combination thereof and an bacteria specific enzymatically digestible substrate.

39. The bioassay module (10) according to claim 33, wherein said food specimen deposit element (5) allows a food specimen to be placed on top of said enzymatically digestible substrate layer and prevents spillage.

40. The bioassay module (10) according to claim 33, wherein said indicator layer (3) is selected from a group consisting, color indicator, light indicator, pH indicator, consistency indicator, electrochemical indicator and any combination thereof.

41. A kit for the detection of pathogenic microorganisms in food comprising:
a. a food specimen deposit element (5),
b. an indicator layer (3),
c. ingredients for the preparation of an enzymatically digestible substrate layer (4),
d. an isolation layer (6),
e. a separation layer (2); and
f. an absorption layer (1),
g. preparation, assembly and operation instructions,
wherein consequent to the preparation of said enzymatically digestible substrate layer according to said instructions, said layers and said food specimen deposit element are assembled and layered from bottom to top, said absorption layer (1), said separation layer (2), said indicator layer (3), said isolation layer (6), said enzymatically digestible substrate layer (4) and said food specimen deposit element (5), once all layers are assembled a food specimen is deposited for the detection of said pathogenic microorganisms.

42. The bioassay kit, according to claim 41, wherein said pathogenic microorganisms are selected from a group comprising Bacillus cereus, Enterococcus faecalis, Pseudomonas aeruginosa, Serratia marcescens, Bacillus amyloliquefaciens, Bacillus subtilis, Bacillus megaterium, Bacillus pichinotyi, Bacillus pumilus, Staphylococcus hominis, Alternaria alternate, Aspargilis ustus, Cladosporium cladosporioides, Penicillium chrysogenum, Aspargilis nidulans, Mucor racemosus, Trichoderma longibrachiatum, Aspargilis versicolor, Cryptococcus albidus and any combination thereof.

43. The bioassay kit according to claim 41, wherein said enzymatically digestible substrate layer (4) is a bacteria specific substrate.

44. The bioassay kit according to claim 41, wherein said enzymatically digestible substrate layer (4) is selected from a group comprising gelatin, agar and PVA (Poly(vinyl alcohol)).

45. The bioassay kit according to claim 41, wherein said enzymatically digestible substrate layer (4) is a gelatin layer.

46. The bioassay kit according to claim 41, wherein said enzymatically digestible substrate layer (4) is a combination of support material selected from a group comprising alginate, sol-gel, gelatin and any combination thereof and an bacteria specific enzymatically digestible substrate.

47. The bioassay kit according to claim 41, wherein said food specimen deposit element (5) allows a food specimen to be placed on top of said enzymatically digestible substrate layer (4) and prevents spillage.

48. The bioassay kit according to claim 41, wherein said indicator layer (3) is selected from a group consisting, color indicator, light indicator, pH indicator, consistency indicator, electrochemical indicator and any combination thereof.

49. A method for the detection of food pathogenic microorganisms comprising:
a. obtaining a bioassay module (10) according to claim 33,
b. depositing food specimen in said food specimen deposit element (5),
c. monitoring said indicator dye on said absorption layer (1).

50. The method according to claim 49, wherein said pathogenic microorganisms are selected from a group comprising Bacillus cereus, Enterococcus faecalis, Pseudomonas aeruginosa, Serratia marcescens, Bacillus amyloliquefaciens, Bacillus subtilis, Bacillus megaterium, Bacillus pichinotyi, Bacillus pumilus, Staphylococcus hominis, Alternaria alternate, Aspargilis ustus, Cladosporium cladosporioides, Penicillium chrysogenum, Aspargilis nidulans, Mucor racemosus, Trichoderma longibrachiatum, Aspargilis versicolor, Cryptococcus albidus and any combination thereof.

51. The method according to claim 49, wherein said enzymatically digestible substrate layer (4) is a bacteria specific substrate or a combination of a support material and said bacteria specific substrate.

52. The method according to claim 49, wherein said food specimen deposit element (5) allows a food specimen to be placed on top of said enzymatically digestible substrate layer and prevents spillage.
